(19) United States
(12) Patent Application Publication
(10) Pub. No.: US 2005/0165322 A1
(43) Pub. Date: Jul. 28, 2005

(54) Method of improving medical apparatus in order to reduce or replace ancillary medical assistance by employing audible verbal human sounding voices which provide therapeutic instructions and encourage usage and give measurements as needed emanating from the apparatus's by using electronic technology

(76) Inventor: Terry Keith Bryant, Singer Island, FL (US)

Correspondence Address: TERRY KEITH BRYANT, 1281 EAST BLUE HERON BLVD, SINGER ISLAND, FL, 33404, US

(21) Appl. No.: 10/767,396

(22) Filed: Jan. 23, 2004

(51) Int. Cl. A61B 5/00; A61B 5/08

(52) U.S. Cl. 600/538; 600/300

(57) ABSTRACT

The present invention relates to an appartus used in the medical industry, in order to increase transpulmonary pressure and respiratory volumes, improve inspiratory muscle performance and re-establish the normal pulmonary hyperinflation, through the employment of electronic technology, providing audible, simulated, verbal, human sounding words, that assist, guide and prompt, increasing patient usage. In the past, lack of usage of this simple plastic, antiquated, disposable unit, by the patient, has contributed to severe problems, such as pneumonia. Without prompting, the patient, finds it hard to inhale into a tube repetitively, to improve their lungs. Previous applications of prior equipment has been poor, thus adding intelligence in the form of electronic technology, which prompts without assistance, is a tremendous advantage in helping not only the sighted, but also the blind as well, since normally only written information accompanies the incentive spirometer, thus, changing the use of this medical device as we know it today.

### BACKGROUND OF THE INVENTION

The present invention relates to enhancement of the Incentive Spirometer Medical Apparatus, through electronic technology to the medical apparatus which is normally used to help in the rehabilitation of the lungs after an operation, or similar type situations. The Incentive Spirometer consist of a plastic bell jar with a float inside the bell that rises, due to air being inhaled through a tube that is attached to the bell jar. By inhaling in the tube, the patient attempts to reach different volumes that are represented on the bell jar, where the float is used as a measuring device, but the float in the bell jar moves slowly and does not remain at it's apogee for very long, making visual accuracy for reading it's measurements on the scale, (on the bell jar), difficult The purpose of this prior art, is to bring air into the patient's lungs. The more air and use of the device, the better the patient's lungs become and thus the lungs are strengthened, however as recent studies have shown, complications such as pneumonia, are due to the lack of compliance, by the patient. Normally, the patient must utilize this medical apparatus without assistance and is expected to basically read written information on how to use the device, which is often performed improperly. Through the improvement of using electronically simulated, audible, verbal, human sounding word, words, or phrases that emanate from within the Incentive Spirometer itself, the ability of this programmed new invention, has the intelligence to detect the patient's measurements, as well as prompting the exact time, that the patient should begin therapy again accordingly. This new improved apparatus, will also give the measurement of the volume that the patient has performed during their therapy, along with encouraging phrases that continue to lead and guide the patient until the full therapy is completed. Prior art required the patient to do the therapy unsupervised and the present invention will provide verbal instruction and guidance electronically, allowing not only the sighted but the blind to benefit as well, providing a new method of technology in the medical industry.

### DESCRIPTION OF THE DRAWINGS

FIG. 1 Shows Preferred Embodiment of Present Invention

A Gauge 2 connects to Audible Response Unit 1 through one or more electrical connections labeled 400.

Audible Response Unit l connects to Speaker 3 through an electrical connection labeled 401.

Power is supplied from Power Supply 4 to Gauge 2 through an electrical connection labeled 402.

Power is supplied from Power Supply 4 to Audible Response Unit 1 through an electrical connection labeled 403.

FIG. 2 Shows the Preferred Embodiment of Audible Response Unit 1 of FIG. 2.

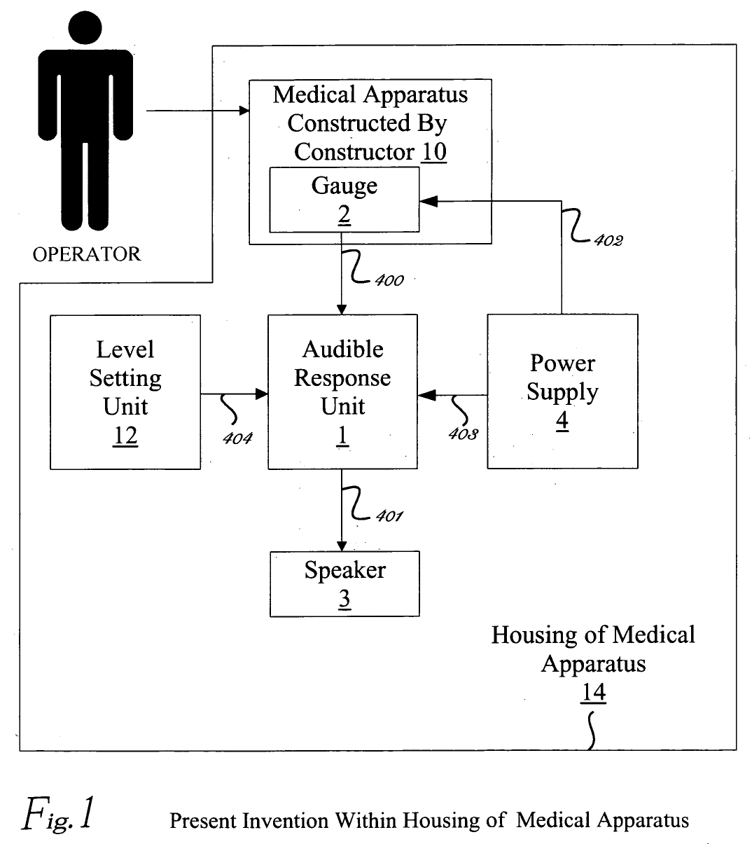

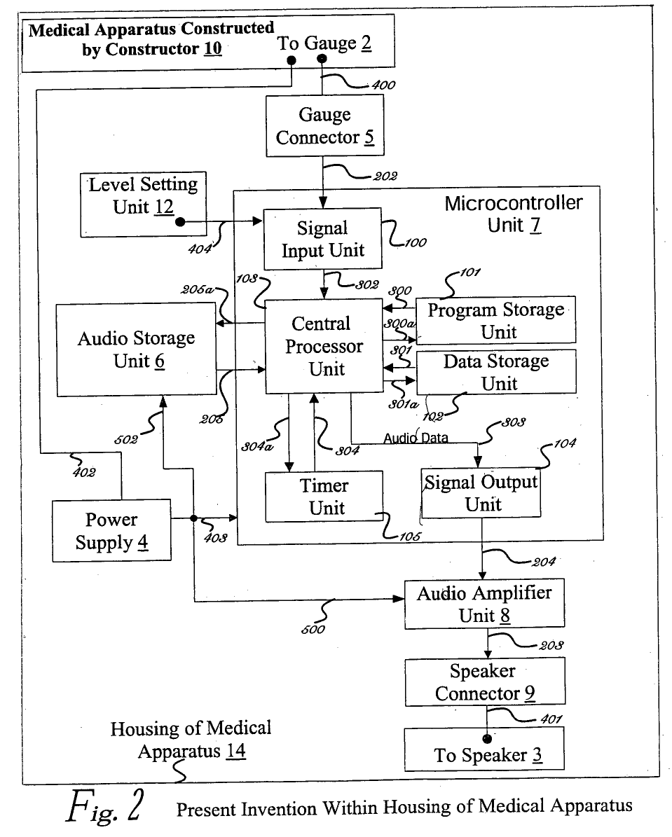

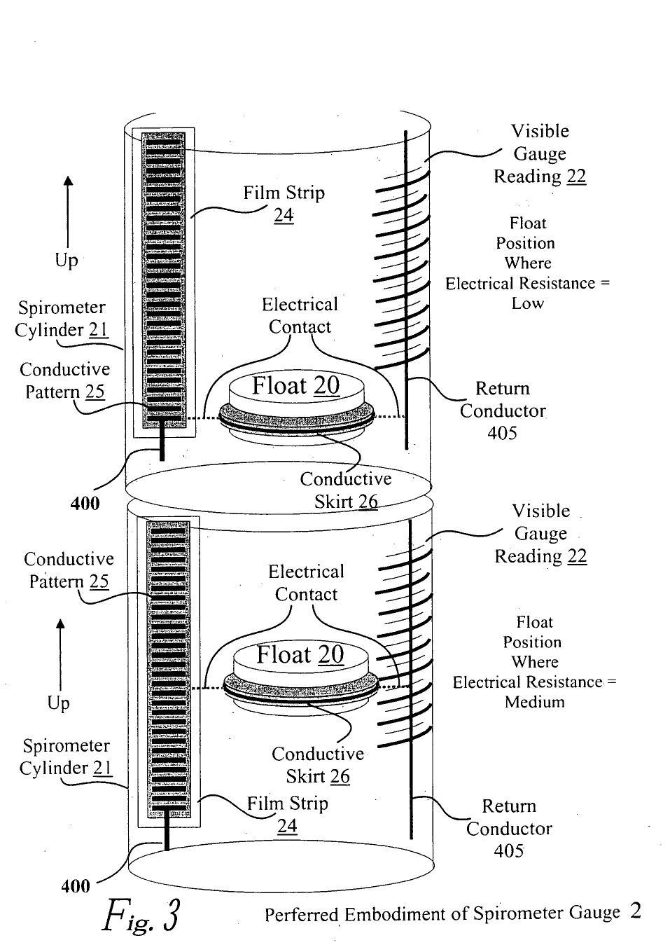

Gauge 2 of FIG. 1 connects to Gauge Connector 5 through one or more electrical connections labeled 400.

Gauge Connector 5 connects to Signal Input Unit 100 which is a subunit of the Microcontroller Unit 7 through one or more electrical connections labeled 202.

Microcontroller Unit 7 contains subunits Signal Input Unit 100, Program Storage Unit 101, Data Storage Unit 102, Central Processor Unit 103, Signal Output Unit 104 and Timer Unit 105.

Signal Input Unit 100 provides information to Central Processor Unit 103 through a set of signals labeled 302.

Central Processor Unit 103 receives a set of program instructions that provide the function of the Audible Response Unit 1 from Program Storage Unit 101 by providing control information through signals labeled 300a and receiving instructions through signals labeled 300. Information used by the program instructions are kept in Data Storage Unit 102 by providing control information and data to be stored through a set of signals labeled 301a and by receiving data through a set of signals labeled 301.

Central Processor Unit 103 controls a set of timers in Timer Unit 105 through a set of signals labeled 304a and receives information from the timers in Timer Unit 105 through a set of signals labeled 304. The Central Processor Unit 103 uses information from Timer Unit 105 to determine accurate time intervals.

Central Processor Unit 103 receives audio data from Audio Storage Unit 6 by providing control information through a set of signals labeled 205a and by receiving audio data through a set of signals labeled 205.

Central Processor Unit 103 relays the audio data received from Audio Storage Unit 6 to Signal Output Unit 104 by transferring the audio data through a set of signals labeled 303. Signal Output Unit 104 transfers audio data to Audio Amplifier Unit 8 through a set of signals labeled 204.

Audio Amplifier Unit 8 transfers amplified audio data to Speaker Connector 9 through a set of signals labeled 203.

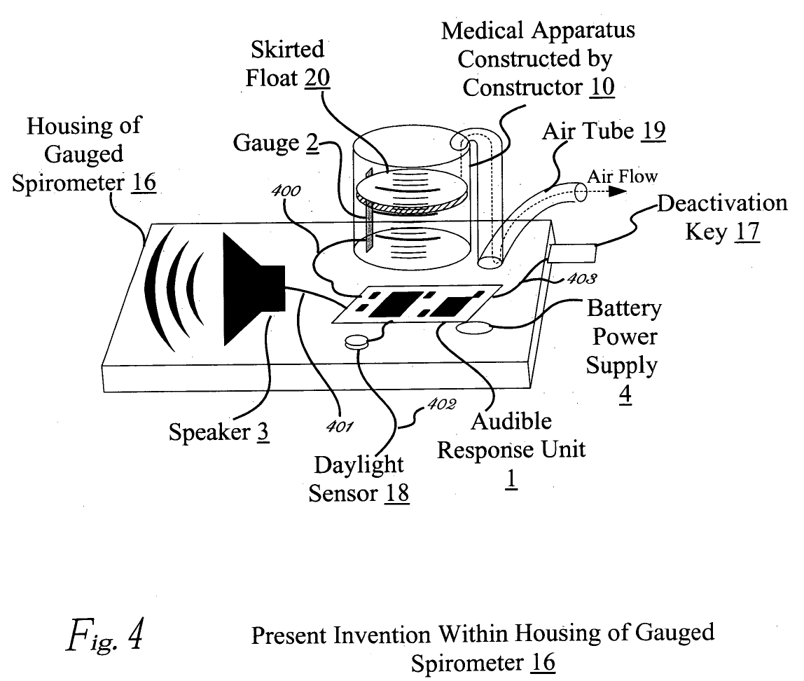

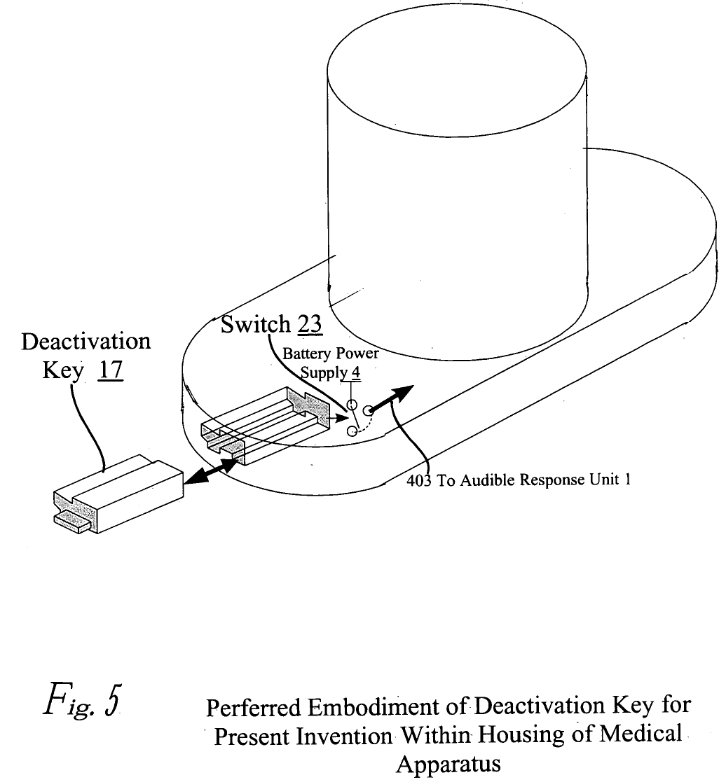

Speaker Connector 9 connects to Speaker 3 of FIG. 2 through a set of signals labeled 401.

FIG. 3 Shows the Present invention within the housing of a Medical Apparatus 10, that implements a Gauged Spirometer whose housing is identified as 16 and which encloses the Medical Apparatus 10, which is comprised of the Speaker 3, Audible Response Unit 1, Battery Power Supply 4, Daylight Sensor 18, and Deactivation Key.

Daylight Sensor 18, is used by the Audible Response Unit 1, that detects that it is nighttime by measuring the signal on 402 and comparing it to a value within the Data Storage Unit 102.

Deactivation Key 17, deactivates the Audible Response Unit 1, that closes a switch that relays a signal over electric conductor 403, comparing it to a value within the Data Storage Unit 102, it enters an operational mode called “silent mode”.

FIG. 4 Detail of Gauge 2, Film Strip 24 is attached to the inside wall of Spirometer Cylinder 21, covered with a Conductive Pattern 25, Float 20 moves freely up and down within the Spirometer Cylinder 21, making contact with Conductive Pattern 25 of Film Strip 24, which is covered with Conductive Skirt 26, this creates a electric path from contact with Film Strip 24 and the Return Conductor 405.

Current from electric conductor 400, through Film Strip 21, through Conductive Pattern 25, through Float Skirt 26, through Return Conductor 405, is proportional to the position of electrical contact, called “float signal”.

“Float Signal” is relayed to Audible Response Unit 1, by electric conductor 400, interpreted in Audible Response Unit 1 and is able to measure and record performance.

FIG. 5 Detail of Deactivation Key 17, which causes switch 23 to close, thus connecting Battery Power Supply 4, to electrical conductor 403, causing a signal on electric conductor 403, relayed to Audible Response Unit 1, interpreting the signal on electrical conductor 403 as described in FIG. 6.

### DETAILED DESCRIPTION OF THE INVENTION

When the Apparatus 10 in FIG. 1 is used by the operator, a Gauge 2 within the Apparatus produces an electrical signal on electrical conductor 400 proportional to the physical parameter that is measured by the Gauge 2. The electrical signal on 400 is variable over time and represents an electrical representation of the parameter measured by the Gauge 2 during the duration of time that the Apparatus 10 is used. The electrical signal on 400 is input to the Audible Response Unit 1 where the electrical signal on 400 is evaluated.

The Gauge Connector 5 on FIG. 2 relays the electrical signal on 400 to the Signal Input Unit 100 within Microcontroller Unit 7 where the electrical signal on 400 is converted repeatedly at a fixed rate of once every unit of time called the “sampling interval” for the duration of time when the electrical signal on 400 is being evaluated. The Signal Input Unit 100 converts the electrical signal on 400 into a digital numerical format and relays it through a set of digital electrical signals 302 to the Central Processor Unit 302. This process is repeated after the transpiring of time equal to the sampling interval for the duration of time over which the electrical signal on 400 is being evaluated.

The parameter being measured by Gauge 2 is thereby converted to a sequence of numerical digital values that represent the magnitude of the parameter over the time duration when the parameter is being evaluated, and each successive numerical digital value represents the magnitude of the parameter measured by Gauge 2 at the time that is one “sampling time” interval later than the preceeding numerical digital value.

The Central Processor Unit 103 executes a sequence of instructions that are retrieved from the Program Storage Unit 101. This sequence of instructions is called the “functional program” and defines the series of steps and decisions that are made to constitute the function of the present invention. The Central Processor Unit 103 retrieves the instructions from the Program Storage Unit 101 by presenting an index called a “program address” to the Program Storage Unit 101 through the set of digital electrical signals 300a. The “program address” is calculated by the Central Processor Unit 103 as directed by the instructions of the “functional program” that it is executing. The Program Storage Unit 101 responds to the “program address” on 300a by retrieving and relaying the instruction corresponding to the “program address” to the Central Processor Unit 103.

The instructions representing the “functional program” relayed to the Central Processor Unit 103 by the Program Storage Unit 101 over digital electrical signals 300a are executed by the hardware within the Central Processor Unit 103 to perform mathematical caculations, “program address” generation, and decision logic which together constitute the “functional program” of the present invention which in turn defines the behavior and function as defined for the Apparatus 10.

Intermediate mathematical and logical calculations that are performed by the Central Processor Unit 103 as it executes the “functional program” result in information collectively called “data” that are stored in the Data Storage Unit 102. The Central Processor Unit 103 identifies storage locations in the Data Storage Unit 102 for storing or retrieving “data” by presenting an index called the “data address” to the Data Storage Unit 102 through a set of digital electrical signals 301a. The Central Processor Unit 103 generates the “data address” by performing calculations that it is directed to peform by the instruction of the “functional program” that is being executed. The Central Processor Unit 103 also presents “data” to be stored through the set of digital electrical signals 301a to the Data Storage Unit 102. If the Central Processor Unit is retrieving data from the Data Storage Unit 102, the Data Storage Unit 102 presents the retrieved data associated with the “data address” on 301a to the Central Processor Unit 103 through a set of digital electrical signals 301.

The Central Processor Unit 103 directs the Timer Unit 105 by presenting commands that are calculated during the execution of the “functional program” to the Timer Unit 105 through a set of digital electrical signals 304a. The commands instruction Timer Unit 105 on the time intervals that are to be generated. The Timer Unit 105 relays time interval information to the Central Processor Unit 103 through a set of digital electrical signals 304. The Central Processor Unit 103 uses the timer interval information for purposes of indicating when one or a set of instructions of the “functional program” should execute. The provides the ability of the Central Processor Unit 103 to synchronize the execution of one or a set of instructions of the “functional program” to a precise point in time or an interval of time.

When the Central Processor Unit 103 determines that an audible response is needed and which audible response is to be generated as determined by the definition of the behavior of the Apparatus 10 and the definition of the “functional program”, it is directed by the instructions within the “functional program” to calculate an index called the “audio address” that is used to retrieve the audible response data called “audio data” from the Audio Storage Unit 6. The Central Processor Unit 103 presents the “audio address” to the Audio Storage Unit 6 through a set of digital electrical signals 205a. The Audio Storage Unit 6 responds by relaying the “audio data” associated with the “audio address” to the Central Processor Unit 103 through a set of digital electrical signals 205.

The Central Processor Unit 103 retrieves time interval information from Timer Unit 105 to determine the appropriate time when retrieved “audio data” can be relayed to the Signal Output Unit 104. In this way, the “audio data” is successively relayed to the Signal Output Unit at a rate appropriate for the regeneration of the audible response from the “audio data”. The Central Processor Unit 103 relays the “audio data” to the Signal Output Unit 104 through a set of digital electrical signals 303.

The Signal Output Unit 104 recieves “audio data” from the Central Processor Unit 103 at a rate that is indicated by time interval from the Timer Unit 105. The time interval is calculated by the Timer Unit 105 as it is commanded to do by the Central Processor Unit 103 when it executes the instructions in the “functional program” that controls setting up of the Timer Unit 105. The time interval is made to be the value required in order to regenerate the audible response correctly when “audio data” is repetitively output at a rate equal to the time interval.

The Signal Output Unit 104 receives “audio data” in a digital numerical form from the Central Processor Unit 103 repetitively starting from the first unit of “audio data” to the last unit of “audio data”. The Signal Output Unit 104 converts the “audio data” to an electrical signal whose magnitude is proportional to the “audio data” repetitively for each “audio data” received. It relays the elecrical signal to the Audio Amplifier Unit 8 through an electrical signal 204. The Audio Amplifier Unit 8 multiplies the magnitude of the electrical signal relayed on the electrical signal 204 such that the amount of power represented by the electrical signal 204 is increased and output to the Speaker Connector 203. The Speaker Connector 9 relays the amplified electrical signal on 203 to electrical signal 401 which corresponds to electrical signal 401 on FIG. 2 The amplified electrical signal 401 is presented to the Speaker 3 in FIG. 2.

The Speaker 3 converts the amplified electrical signal 401 to sound energy that represents the audible response that the Audible Reponse Unit 1 has calculated in response to the measurement of a parameter that is determined by the Gauge 2 of the Apparatus 10 in accordance to the defined behavior of the Apparatus 10 and of the defined function of the “functional program.”

The present invention describes a method of producing audible response to the measurement of a parameter by an Apparatus 10 so that the audible response is done according to a defined behavior determined by the constructor of the Apparatus 10. Implementation of the defined behavior of the audible response to measurement of a parameter within the Apparatus 10 is realized by the defined function of the “functional program” that is coupled to the Audible Response Unit 1 by storing the “functional program” in the Program Storage Unit 101 within the Audible Response Unit 1 and by providing a means for the Central Processor Unit 103 within the Audible Response Unit 1 to execute the instructions in the “functional program” and to peform the actions as they direct the Central Processor Unit 103 and the other subunits within the Audible Response Unit 1.

FIG. 3 shows the Present Invention within the housing of a Medical Apparatus 10 that implements a Gauged Spirometer whose housing is identified as 16 and which encloses the Medical Apparatus 10 as well as the present invention which is comprised of the Speaker 3, Audible Response Unit 1, Battery Power Supply 4, Daylight Sensor 18, Deactivation Key 17. The Medical Apparatus in this embodiment is constructed to perform Spirometry measurements of the medical patient referred herein as the “operator”. In this embodiment of the present invention, the Power Supply 4 is implemented as a Battery in order to provide a means of operating the Medical Apparatus without the need to connect to an auxiliary power source through means of wire cords. This means is referred to as using a “cordless” power supply.

The present invention also includes a Daylight Sensor 18 that is used by the Audible Response Unit 1 to distinguish between daytime and nighttime. The Daylight Sensor 18 is constructed as but not limited to a photocell that relays a signal to the Audible Response Unit 1 over electrical conductor 402. When the Audible Response Unit 1 detects that it is nighttime by measuring the signal on 402 and comparing it to a value within the Data Storage Unit 102, it enters an operational mode called “silent mode”. In “silent mode”, the Audible Response Unit 1 activates itself at the same time intervals as it does in daytime, but does so in order to measure the daylight by means of sensing the Daylight Sensor 18. If sufficient daylight is not detected, the Audible Response Unit 1 does not emit any audible instructions to the operator but instead sets an internal timer to reactivate itself after a prescribed time interval that is defined in the “functional program” of the Audible Response Unit 1 and then deactivates itself. With this method of daytime detection, it is possible for the Audible Response Unit 1 to permit the “operator” to rest during the nighttime and to maintain a regular programmed interval for reactivation. When the Audible Response Unit 1 is reactivated at the transpiring of the programmed time interval as defined in its “functional program” and detects sufficient daylight, the Audible Response Unit 1 enters an operational mode called “standard mode” and begins emitting audible commands to the “operator” as defined by the “functional program” within the Audible Response Unit 1.

The present invention also includes a Deactivation Key 17 that provides to the means to deactivate the Audible. Response Unit 1 for any period of time in the event that such deactivation is determined to be necessary by qualified personnel responsible for the medical care of the “operator”. The Deactivation Key 17 is a mechanically unique shape that matches the same mechanically unique cavity within the Housing of the Gauged Spirometer 16. The Deactivation Key 17 when inserted into the housing of the Gauged Spirometer 16 closes a switch that relays a signal over electrical conductor 403 to the Audible Response Unit 1 to indicate the presence of the Deactivation Key 17. When the Audible Response Unit 1 detects that the Deactivation Key 17 is present by measuring the signal on 403 and comparing it to a value within the Data Storage Unit 102, it enters an operational mode called “silent mode”. In “silent mode”, the Audible Response Unit 1 activates itself at the same time intervals as it does in “standard mode”, but does so in order to measure the presence of the Deactivation Key 17 by sensing the signal on 403. If the Deactivation Key 17 is is determined to be present, the Audible Response Unit 1 does not emit any audible instructions to the operator but instead sets an internal timer to reactivate itself after a prescribed time interval that is defined in the “functional program” of the Audible Response Unit 1 and then deactivates itself. With this method of detection of Deactivation Key 17, it is possible for the Audible Response Unit 1 to permit the qualified personnel to deactivate the Audible Reponse Unit 1 for any period of time and to maintain a regular programmed interval for reactivation. When the Audible Response Unit 1 is reactivated at the transpiring of the programmed time interval as defined in its “functional program” and detects the absence of the Deactivation Key 17, the Audible Response Unit 1 enters an operational mode called “standard mode” and begins emitting audible commands to the “operator” as defined by the “functional program” within the Audible Response Unit 1.

FIG. 4 shows a detail of Gauge 2 as constructed for the Spirometry application show in FIG. 3 The Gauge 2 is constructed of a thin Film Strip 24 of resistive material typically consisting of but not limited to carbon or graphite. The Film Strip 24 is attached to the inside wall of the Spirometer Cylinder 21 with adhesive. The surface of the Film Strip 24 that faces the interior of the Spirometer Cylinder 21 is covered with a Conductive Pattern 25. The Float 20 is free to move up and down within the Spirometer Cylinder 21 and makes contact with the interior facing surface's Conductive Pattern 25 of Film Strip 24 at a point that corresponds to the height position of the Float 20. The outer edge of the Float 20 that contacts the interior facing surface of the Film Strip 24 is covered with a Conductive Skirt 26. The Conductive Skirt 26 creates an electrical path from the position of contact with the Film Strip 24 and the Return Conductor 405. The Float 20 rises as the “operator” inhales through the Air Tube 19 of FIG. 6 so that the gas pressure above the float is lower than the gas pressure beneath the float which is at standard 1 atmosphere. The Float 20 ceases rising when the difference between the gas pressure above and beneath the Float 20 multiplied by the cross sectional surface area (in the direction of the axis of the Spirometer Cylinder 21) of the Float 20 is equal than the weight of the float 20. The Float 20 falls when the difference between the gas pressure above and beneath the Float 20 multiplied by the cross sectional surface area (in the direction of the axis of the Spirometer Cylinder 21) of the Float 20 is less than the weight of the Float 20.

The amount of electrical current flowing from the electrical conductor 400 through the Film Strip 21 through Conductive Pattern 25 through the Float Skirt 26 through the Return Conductor 405 referred to as the “float signal” is proportional to the position of the electrical contact between the Conductive Pattern 25 and the Float Skirt 26 referred to as the “contact point”. The higher the “contact point” is, the more distance there is between the electrical conductor 400 and the “contact point” and hence the more resistive material that comprises the Film Strip 21 there is, and the higher the electrical resistance there is to current flow from electrical conductor 400 to the Return Conductor 405. The position of the contact point corresponds to the height position of the Float 20. Therefore, the amount of electrical current of the “float signal” through electrical conductor 400 is proportional to the height position of the Float 20. The higher the position of the Float 20, the less electrical current there is flowing through the electrical conductor 400 at the “float signal”. The lower the position of the Float 20, the higher the electrical current there is flowing through the electrical conductor 400 at the “float signal”.

The “float signal” is relayed to the Audible Response Unit 1 by electrical conductor 400 and is interpreted by the “functional program” in the Audible Response Unit 1. The Audible Response Unit 1 takes measurements of the “float signal” and determines the level of the signal that corresponds to when the Float 20 reaches it's apogee and when it settles back down to the bottom of the Spirometer Cylinder. By making this determination, the Audible Response Unit is able to measure and record the performance of the “operator” as measured by the Spirometer.

FIG. 5 shows a detail of an example of embodiment of the Deactivation Key 17. It is comprised of a uniquely mechanically shaped device that fits precisely into a cavity within the Housing of the Gauged Spirometer 16. When successfully inserted into this cavity, the Deactivation Key 17 causes switch 23 to close thereby-connecting the Battery Power Supply 4 to the electrical conductor 403. The connection of the Battery Power Supply 4 through switch 23 causes a signal on electrical conductor 403 that is relayed to the Audible Reponse Unit 1. Audible Reponse Unit 1 interprets the signal on 403 as described in the previous description of FIG. 3

I. Electronic technology which has been especially developed to work within the incentive spirometer, that will help the patient by providing simulated audible, verbal, human sounding voices, thus providing instructions, prompting appropriate usage according to therapeutic time schedules, correcting and encouraging patient performance, as well as, giving the appropriate measurement, that the person or patient has performed with the apparatus, eliminating human visual error, help assist the blind and the visually impaired, though the use of today's state of the art equipment, that can produce electronic intelligence within the apparatus at a low cost, thus reducing patients recovery time and complications,
1) a method of providing audibly and verbally, instruction and guidance, to help perform the therapeutic sessions by the patient to improve lung performance, which through medical studies has shown that very few patients perform the required therapy as suggested though the accompanied literature, but through the usage of the present invention, the percentage in regards to lung problems occurring due to failure of patient usage of the incentive Spirometer, will decrease dramatically as the present invention will nag or prompt the patient without stopping, until the patient uses the apparatus and will not stop until the time interval necessary to fulfill the patient's therapeutic need has been accomplished. Through electronic intelligence, the present invention, will prompt the patient to use the medical apparatus, as well as, guide the patient through the proper steps of using said medical apparatus, thus quicker patient recovery will be achieved, through compliance without complication,
2) replacing the normal human visual readings or measurements, eliminating human error of inaccurate readings, due to the prior required float recognition which is imperative to provide visual measurement, since the float doesn't stay always in position long enough to read properly and has to be constantly viewed during therapeutic sessions to observe the exact reading of measurement, with a human sounding electronically programmed voice or voices giving the same readings or measurements as deemed necessary to provide the sighted, as well as the visually impaired patient, with adequate information, to fulfill the patient's therapeutic regiment for recovery and allowing the blind to hear and respond, to the full operation of the therapeutic regiment, of the present invention;
3) a medical apparatus that because of the inexpensive construction, is comparable to the same concept, in relationship to therapeutic use, as the expensive apparatus, due to today's advanced technology. This breakthrough in modern technology allows the patient to afford the new improved apparatus of the present invention, which basically supplies all of the same healthcare purposes in relationship to the therapy of the apparatus, however, it also gives the patient the advantage of hearing the therapeutic guidance and measurements as an added benefit and cost is virtually the same as most disposable incentive spirometry units;

II. A new method to provide the above function of the present invention through the following electronic technology:
1) a number of the following electronic components in order to provide the function as above claimed:
(a) one or more electronic sensors producing an output signal,
(b) one or more electronic modules that convert said sensor output signal (s) into digital format,
(c) one or more electronic modules that includes but is not limited to a central processing unit,
(d) one or more electronic modules for digital storage of program instructions and data,
(e) one or more electronic modules for digital storage of digital audio sound data,
(f) one or more electronic modules for generation of audible sound,
(g) one or more electronic modules for managing and conserving electrical power,
(h) one or more electronic modules for determining accurate intervals of time,
(i) one or more electronic modules for communicating remotely with separate agent,
(j) one or more electronic sensor for detecting light or the absence of light to turn off or on unit
2) said method of new apparatus capable of measuring output signal of the sensors, converting said output signals into digital format to be stored and processed by the central processing unit, resulting in actions taken by the central processing unit under direction of it's digital program instructions in accordance to it's predetermined set of actions,
3) said pre-determined actions of the digital program instructions include but not limited to the generation of audible audio sound sequences that provide information relating to said output signals,
4) said electronic sensors capable of measuring but not limited to parameters of performance of the human body in various settings relating to medical therapeutic performance, or physical training,
4a) said electronic sensors being comprised of, but not limited to, a resistor that forms a variable resistance to electric current flow, such as a film of carbon, but not limited to, that forms a resistance to electric current flow, in contact with said resistor,
5) said central processing unit capable of performing tasks as specified in the order defined in digital program, including, but not limited to processing of sensor output signals, execution of control functions defined by the digital program, providing actions in accordance to accurate time intervals, generation of audible sound,
6) said digital program defines control functions that implement therapy or physical rehabilitation regimes,
7) said digital program defining control functions that implement tasks for managing and conserving electrical power,
8) said digital program defining control functions that implement tasks for determining accurate intervals of time,
9) said digital program defining control functions that implement tasks for determining time of day, (for those medical apparatus that need to be turned on or off to begin or end therapeutic sessions),
10) said digital program defining control functions that implement tasks for communicating with a separate agent,
11) said digital program being stored in memory within the electronic module that contains the central processing unit, and or being stored in memory that is not within the electronic module that contains the central processing unit but that is accessible by the central processing unit,
12) said digital audio sound data being stored in memory within the electronic module that contains the central processing unit, and or being stored in memory that is not within the electronic module that contains the central processing unit but that is accessible by the central processing unit,
13) directory table containing descriptive information about those commands, responses, measurements, or words as aforementioned about said digital audio sound data that is stored in memory within the electronic module that contains the central processing unit, or being stored in memory that is not within the same electronic module that contains the central processing unit but that is also accessible to the central processing unit,
13a) said digital audio sound data being arranged into multiple units, each unit representing an audible verbal message comprised of a series of words as programmed per the requirements in synthesis with the medical apparatus's therapeutic use,
13b) a method for retrieving and generating the audible sound representing the digital audio data from the start of the message to the end of the message as corresponds to the therapeutic dialogue needed,
13c) a method for retrieving and generating the audible sound representing the digital audio data from an intermediate point in the message to a subsequent intermediate point in the same message, to allow the medical apparatus to respond to the measurements being produced by the patient accordingly and guide the patient according to the measurement amount,
14) said electronic module for generation of audible sound being the same electronic module that contains the central processing unit, and or a being separate electronic module for the module that contains the processing unit,
15) said electronic module for generation of audible sound including a module that converts digital audio data into continuous analog signal that is amplified to increase the signal power as needed to create audible sound from sound generating modules such as, but not limited to, speakers,
15a) said electronic modules for generation of audible sound providing a sound generating a continuous analog signal that is one half the value of the maximum signal level, such level representing zero sound to be generated,
15b) said electronic module for generation of audible sound providing a sound generating module such, but not limited to, speaker(s) that is capable of receiving a level that is one half the maximum signal level in a way that produces no sound and consumes little or no power,
15c) said sound generating module such as, but not limited to, a speaker(s) whose reference signal level is set at one half the maximum signal level such that it produces no sound when it receives such a signal level,
15d) said sound generating module being provided a reference signal level set at on half the maximum signal level by connecting it between a series of batteries in a way that provides a reference signal that is exactly on half the signal level that is produced by the above said batteries connected in this way,
16) said digital program defining a method for determining the value of a sensor output signal, generating an audible verbal response according to a pre-determined set of controls and functions as described herein, in order to provide instructional information to the operator of whatever medical apparatus is being used for instructional information or guidance,
17) said digital program defining a set of predetermined set of controls and functions relating sensor output signals to audible verbal commands, responses and measurements, comprises of improving medical conditions of the patient through the use of the said medical apparatus accordingly, along with the present invention.
